(19) United States
(12) Patent Application Publication
(10) Pub. No.: US 2023/0375522 A1
(43) Pub. Date: Nov. 23, 2023

(54) METHOD FOR ANALYZING SOIL POLLUTION

(71) Applicant: Tellux, Saint- Paër (FR)

(72) Inventors: Antonin Van Exem, Saint- Paër (FR); Paul Honeine, Rouen (FR); Melanie Mignot, Canteleu (FR)

(21) Appl. No.: 18/246,842

(22) PCT Filed: Sep. 28, 2021
(86) PCT No.: PCT/FR2021/051667

§ 371 Date: Mar. 27, 2023

(30) Foreign Application Priority Data
Sep. 30, 2020 (FR) 2009996

(51) Int. Cl. G01N 33/24; G01N 21/85; G01N 21/94

(52) U.S. Cl. CPC G01N 33/24; G01N 21/8507; G01N 21/94

(57) ABSTRACT

A method for analyzing soil contamination by pollutants, in particular, organic pollutants, using hyperspectral analysis of reflection and/or photoluminescence, is characterized in that analysis is carried out by illuminating a sample using a first item of equipment provided with a light source and at least one spectral sensor sensitive to a spectrum ranging from thermal infrared to ultraviolet.

### CROSS-REFERENCE TO RELATED APPLICATIONS

This application is a national phase entry under 35 U.S.C. § 371 of International Patent Application PCT/FR2021/051667, filed Sep. 28, 2021, designating the United States of America and published as International Patent Publication WO 2022/069827 A1 on Apr. 7, 2022, which claims the benefit under Article 8 of the Patent Cooperation Treaty to French Patent Application Serial No. FR2009996, filed Sep. 30, 2020.

### TECHNICAL FIELD

The present disclosure relates to the field of analyzing soil contamination by organic pollutants.

### BACKGROUND

The sources of soil contamination are varied: the use of fertilizers, pesticides, waste from an industrial site, the proximity of an incinerator, a waste storage site, waste from drug residue from farm animal excretions, hydrocarbons, etc. The pollution of soil, fields, gardens or playing fields may originate from chemical agents: dioxins, PCBs or toxic metals that are extremely dangerous for health.

The origins of this pollution may be accidental (isolated spills or deposits of pollutants due to neglect, malfunction of an industrial facility, accident of a plant or a vehicle for transporting polluting materials), with a large amount of pollutant discharged, or chronic (continuous supply of contaminants by leakage or leaching, the cumulative effects of which may be greater and more insidious than those of accidental pollution).

Polluted soil may be the cause of poisoning when consuming garden fruits or vegetables. The bioaccumulation of pollutants contained in soil in plants and animals makes these the pollutants that are most dangerous for human and animal health. Indeed, they have the particularity of contaminating the food chain (dioxins, PCB, radioactivity, etc.). Pollutants contained in soil may also cause irritations of the skin and the respiratory system. They are also responsible for cardiac and neurological disorders, loss of fertility, fetal development disorders, and are the cause of certain cancers.

To evaluate the presence of contamination and to qualify and quantify the nature of the pollutants, it is common practice to take samples, for example, by core sampling, and to submit these samples to a physicochemical analysis laboratory. The “Guide méthodologique pour l'analyse des sols pollu\tilde{e}s” [“Methodological guide for the analysis of polluted soil”] published in February 2000 under reference BRGM/RP-50128-FR presents in detail the techniques for analyzing polluted soil.

Since these analyses require high-level scientific resources and skills, it has also been proposed to automate all or part of these analyses.

For a given site, the conventional soil analysis steps involve the following:
1. contracting a surveying company to take the core samples;
2. sending samples to analysis laboratories;
3. waiting for the analyses to return;
4. interpreting these analyses as maps;
5. drawing up a recommendation report.

The minimum duration of such an analysis is from 6 to 8 weeks. Following these analyses, the report is often at risk of showing significant uncertainties due to the heterogeneity of the soil studied and thus of recommending a new sampling campaign. Indeed, the initial sampling is often insufficient since the distances between sampling points are too large (cost policy). It is then necessary to restart the loop described in the above-mentioned problem one or more times. The delay due to this problem is at least one month.

Finally, during pollution removal, the field is dug up by the pollution control company and unexpected pollution can be detected. This leads to stopping the work, and resampling and analysis as described above are restarted. During this time, the excavated materials are temporarily stored on the site before the nature of the pollution is known and they can be sent to the proper reprocessing centers. In this case, an additional delay of at least 15 days is observed, in addition to significant cost overruns related to the immobilization of workers and machines, as well as the reprocessing of the polluted earth not initially diagnosed.

In the state of the art, the article by Bin Zou, Xiaolu Jiang, Huihui Feng, Yulong Tu, Chao Tao, “Multisource spectral-integrated estimation of cadmium concentrations in soil using a direct standardization and Spiking algorithm” published in Science of The Total Environment, Volume 701, 2020, 134890, ISSN 0048-9697 is known, relating to the field of low-level and satellite remote sensing on a large scale and more precisely the study of the exact spectral response of cadmium (Cd) in the soil, and presents a novel method by combining direct standardization (DS) and Spiking algorithms to integrate multisource spectra in order to improve the accuracy in estimating the Cd concentration.

This article relates to the analysis of the presence of a heavy metal, cadmium, in samples in which it is present. This article does not provide any teaching on the characterization of unknown pollutants, in particular, organic pollutants, in a sample taken by core sampling in the field.

The publication “Scafutto, Rebecca & Souza Filho, Carlos. (2016). Quantitative characterization of crude oils and fuels in mineral substrates using reflectance spectroscopy: Implications for remote sensing. International Journal of Applied Earth Observation and Geoinformation. 50. 221-242. 10.1016/j.jag.2016.03.017.” is also known, relating to the environmental monitoring of oil and fuel leaks using proximal and far range multi spectral, hyperspectral and ultraspectral remote sensing.

This publication is based on measuring the near and shortwave infrared spectral reflectance properties of several mineral substrates impregnated with crude oil, diesel, gasoline and ethanol by means of Principal Component Analysis (PCA) and Partial Least Square (PLS) regression. These features were used for the qualitative and quantitative determination of the contaminant impregnated in the substrates. Specific wavelengths, where key absorption bands occur, were used for the individual characterization of oils and fuels. The intensity of these features can be correlated to the abundance of the contaminant in the mixtures. Grain size and composition of the impregnated substrate directly influence the variation in the spectral signatures.

Finally, the article “Kopel, Daniella & Brook, Anna & Wittenberg, Lea & Malkinson, Dan. (2015). Spectroscopy as a Diagnostic Tool for Urban Soil. Water, Air, and Soil Pollution. 226. 10.1007/sll270-015-2442-2.” is known, regarding the detection of spectral activity (SA) in a structured hierarchical approach to identify dominant spectral features.

The developed method is adopted by multiple in-production tools, in particular, continuum removal normalization, guided by polynomial generalization, and spectral likelihood algorithms: orthogonal subspace projection (OSP) and iterative spectral mixing analysis (ISMA).

The solutions of the prior art do not make it possible to achieve a sufficient level of precision and reliability when the analysis is carried out directly on the site by a spectrometric method and, in particular, by hyperspectral imaging.

Furthermore, in order to meet the field needs, it is important to be able to provide qualified information on the content of pollutants based upon depth, in order to be able to optimize the treatment of the field, and solutions based on the analysis of remotely collected images are unsuitable.

### BRIEF SUMMARY

In order to solve these disadvantages, the present disclosure relates in its most general sense to a system for analyzing soil contamination by pollutants, in particular, organic pollutants, including reflection spectroscopy equipment, characterized in that the equipment is a portable item of equipment including a light source, in particular, a xenon or halogen source, and at least one spectral sensor.

It also relates to a method for analyzing soil contamination by pollutants, in particular, organic pollutants, by means of hyperspectral analysis of the reflection and/or photoluminescence, characterized in that the analysis is carried out by means of a first item of equipment by illuminating a sample using a light source and by at least one spectral sensor sensitive to a spectrum ranging from near infrared NIR to ultraviolet UV. “Near infrared NIR” is understood to mean the wavelength range from 0.78 to 2.5 μm.

Advantageously, the spectral sensor is sensitive over a wider range, including medium-wavelength infrared (MWIR) and/or long-wavelength infrared (LWIR) as well as short-wavelength infrared (SWIR).

The method according to the present disclosure includes:
a learning sequence comprising analyzing a plurality of reference samples, and recording in a learning database:
   a) the spectral signature of reflection acquired by spectral analysis;
   b) known values of the variables representative of the contaminants present in each of the reference samples;
   c) known values of the variables representative of the substrates of each of the reference samples;
a sequence for calibrating an item of field analysis equipment with respect to the first item of equipment, the item of field equipment including a light source and a spectral sensor,
sequences for analyzing a soil sample of a geological site comprising acquiring the reflection and/or photoluminescence signature of the sample using the item of field equipment thus calibrated,
and estimating the characterization of the pollutants by processing the signature by a learning engine exploiting the data from the database established during the learning sequence.

Advantageously, the analysis system according to the present disclosure includes a probe including at least one optical fiber for transmitting light between the analysis zone of the probe on the one hand, and the light source, in particular, a xenon or halogen light source, and the at least one sensor on the other hand.

According to an optional variant, the item of equipment further includes a physicochemical analysis means.

### BRIEF DESCRIPTION OF THE DRAWINGS

The present disclosure will be better understood on reading the following description, with reference to the appended drawings relating to non-limiting embodiments, in which:

FIG. 1 depicts the hardware architecture of an example implementation of the present disclosure;

FIG. 2 depicts a block diagram of the present disclosure;

FIG. 3 depicts the functional architecture; and

FIG. 4 depicts the optical diagram of an alternative embodiment of the optical system.

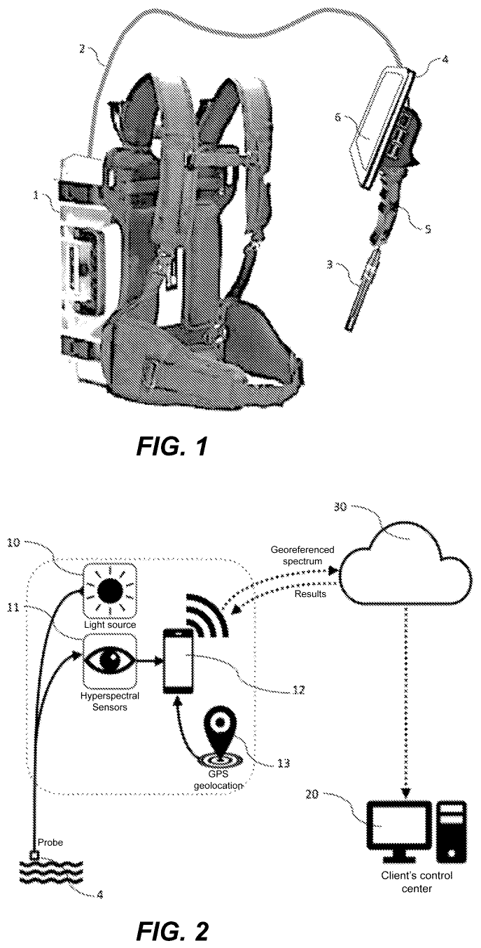

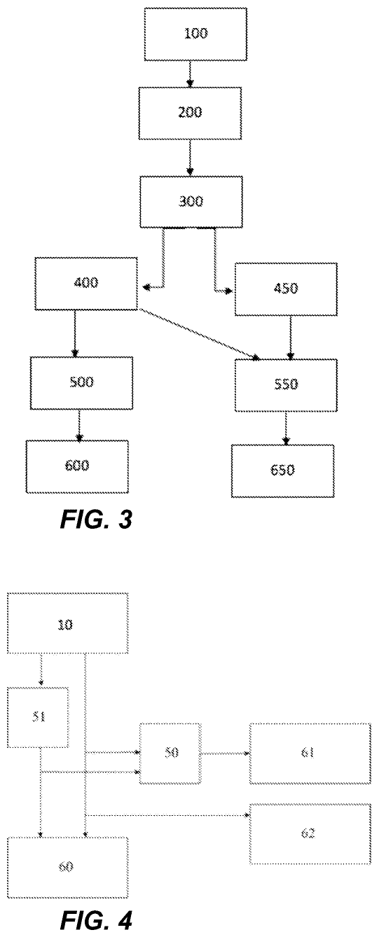

### DETAILED DESCRIPTION

The present disclosure relates to the characterization of soil samples by core sampling in order to determine the qualitative and quantitative presence of constituents of interest (pollutants, in particular, plastics, hydrocarbons, metals, etc.) so as to provide a tool for measuring in real time the nature of the pollutants on sites for reclaiming polluted earth.

The aim is to offer a solution in order to provide, if possible, in real time and on-site, a detailed map of the pollution in order to facilitate and secure the implementation of selective sorting of the excavated materials based upon their level of pollution, directly on the site by virtue of a real-time measurement tool. The result is a three-dimensional map of the field in order to provide geolocated pollution information, in the X and Y coordinates with a resolution of a few square meters to a few hundred square meters, and in depth Z, and then to make it possible to decide with absolute safety on the optimal measures for pollution removal. Optionally, the analysis of samples by core sampling may be completed by an additional remote analysis, by a hyperspectral camera, in order to quantify the pollutants delivered to analysis centers so as to monitor, in real time, the polluted earth that they receive. Thus, the proposed system ensures the quality of the earth and identifies its pollutants in order to allow for its recycling.

For this purpose, the present disclosure enables the instrumentation of boring machines and construction machinery to make it possible to integrate real-time analyses and speed up the process by eliminating the iterative steps and the sources of financial uncertainties.

Thus, when pollution that was not initially identified is detected, real-time measurements would make it possible to direct the excavated material toward the corresponding reclamation line without the laboratory analysis delay (optimization of real-time decision-making operations).

Hardware Architecture

The equipment according to an exemplary implementation of the present disclosure is made up of an item of field equipment (1), which is portable in the example described, including a housing of the backpack type including a source with a broad light spectrum, for example, a xenon lamp (10), as well as a power supply, for example, batteries. The light source (10) is associated via an optical fiber (2) to a lance (3) that transmits light toward the ground and reflects light toward sensors (11), the electrical signals of which are transmitted to a computer that can be housed in the housing or attached in the form of a tablet (4) to the handle (5) of the lance (3).

The sensors are hyperspectral sensors having a sensitivity range between 100 microns and 200 nanometers. These may be a hyperspectral camera, or a multispectral camera, or a set of sensors forming a composite multispectral sensor.

The lance includes one or more optical fibers for transmitting the light emitted by the source (10) to a measurement end, and for collecting the reflected light or the fluorescence light from the sample toward the end of the lance (3). The fiber may include a lens or collimating optics at its end.

The tablet (4) includes a touch interface (6) provided with a geolocation module (13) (GPS or Galileo) and a 5G mobile SIM card.

This equipment further includes a computer (12) and radiocommunication means that make it possible to process the data and provide in real time a diagnosis of the state of the soil and of the pollution (lithological characteristics, type of pollutants, amount of pollutants, 3D map of the soil, etc.) on the one hand, and to communicate the data to a cloud storage space (30) on the other hand. The data are available locally for real-time decision-making but also via an online platform so as to allow the project manager, equipped with a connected terminal (20), present in the control center, to follow the live operations and to collect the data acquired in the field.

Implementation of the Present Disclosure

Core sampling is carried out by an operator equipped with the aforementioned equipment, on a site suspected of contamination, the location of the bore is probed by core sampling or tapping by an instrument provided with a geolocation module, and its geographical coordinates are recorded. Several sections with a depth of about 1 meter are collected in line with the bore and several boring operations are carried out on each site.

All the cores are subjected to spectral analysis. For this, the half-cores, split in two lengthwise, are removed, and data are acquired simultaneously on several half-cores, which is possible due to the rapid imaging acquisition speed. The extraction of sub-images corresponding to a half-core is facilitated by the positioning of QR codes that are digitally recognized and arranged on the corners of the sections. The imaging can be carried out on-site with a camera in a vehicle or a container. A pre-trained predictive model can be used at this stage, on the basis of a sufficient amount of data in the database in order for the pre-trained model (pre-trained on these data) to be sufficient to produce a diagnosis in real time.

The selection of sub-samples is carried out on the basis of the imaging data. Statistical, supervised (machine learning) or non-supervised (end-members extraction, features extraction, novelty detection, etc.) methods are used to select the zones from which these sub-samples are taken in order to depict the heterogeneity of the geological formations present on the site. Spectrometer data may be used alternatively, the collection of the sub-samples may be carried out immediately (in the case of volatile pollutants) or carried out later in the laboratory (the half-cores are resealed hermetically with plastic film and stored in a cold room for preservation).

The chemical analyses comprises carrying out the extraction of pollutants by different methods, by solvent (water, hexane, ether), by agitation, by solid-phase micro-extraction, by microwave, by headspace, etc. The analysis techniques carried out are chromatography (ICP-AES, ion, HS-GS/MS). The analyses are carried out on-site (in a container) or in the laboratory, on sub-samples of cores or samples that are provided and/or non-selectable (e.g., taken by an auger).

The predictive model training is carried out on the basis of a database and reference samples. A first processing model comprises converting raw data originating from the sensor by reflection. This step is referred to as normalization, referring to the current method that uses the raw data measured on a reference material of reflection >99% (Spectralon®) and electronic noise data measured without light source (source) in order to standardize the data of the samples between these two spectra (i.e., 0 and 100% reflection). In order to eliminate these measurements upstream of the acquisitions, a model is generated on the basis of the prior recording of these raw reference data. Raw data measured on 8 reference materials (from 2% to 99% reflection) and electronic noise are used, a model may be trained for each combination of parameters of an apparatus. A second processing level predicts the variables of interest (soil composition, presence of pollutants and amount of pollutant) on the basis of the reflection of a sample. Several training databases are used: published training bases (spectral library of pure compounds, e.g.: USGS Spectral Library), data produced on artificial samples produced in the laboratory or data produced on samples analyzed in the laboratory. In the case where a batch of analyzed samples originates from one site in particular, a model can be trained on this batch only or this batch can be used to improve a pre-trained model on an existing basis by a transfer learning method.

The data processing applies to the imaging data; the data originating from analyzed sub-samples make it possible to interpret or refine a first interpretation of the cores. The model generated with the spectrometer data makes it possible to produce real-time analyses, including analyses referenced with respect to data from COFRAC certified laboratories. The coupling of on-site imaging and spectrometry responds to the phase of diagnosis of a site, and then to the work phase. It is possible to selectively sort the excavated earth according to its waste class. Quality control of the recycled earth at the storage center is carried out on the basis of supplied artificial or semi-artificial samples (sample supplied diluted or artificially doped to cover a higher concentration range and improve the model).

The mapping of the results constitutes a third level of interpretation on the basis of a machine learning model. An interpolation in space of the variables of interest measured on different bores makes it possible to generate a map. The modeling methods of the Gaussian processes are used. The geophysical data measured at the time of boring are used in the mapping by data-merging methods.

In situ measurements may be envisaged. The acquisition of data by fiberized spectrometer makes it possible to probe the soil and to produce a profile of the variables of interest based upon the depth. (Note: a distinction is made between in situ and on-site: the imaging can be carried out on-site with a camera in a vehicle or a container. The term “in situ” relates to the operation of equipment comprising a fiberized probe with one or more spectrometers in the portable version described above).

Block Diagram

The first step (100) comprises taking a sample of a core of a length determined by the depth of ground to be analyzed using the probe (4). The core sampling is geolocated by the GPS module (13) of the equipment.

The next step (200) comprises analyzing the core over the length of the core by measuring the hyperspectral reflection measured by the sensors (11) during illumination by means of the source (10).

The data obtained during the analysis step are recorded locally and in the cloud, and then undergo a step (300) of selecting a subset of samples to carry out either a training of the model (steps 400, 500, 600), or an on-site analysis (steps 450, 550, 650).

Variant of the Learning Step

The learning can be mutualized on the basis of laboratory analyses, with one item of equipment, provided with a high-performance hyperspectral camera, for recording the spectral signatures of a large number of reference samples, and providing a database accessible to a plurality of items of field equipment provided with lower-performance, less expensive sensors.

In order to take into account the technical and optical differences, each item of field equipment is calibrated using reference samples, the spectral signature of which has previously been recorded in the database. A correction function is computed, making it possible to exploit the content of the database with an item of equipment different from that used for the initial analysis.

The samples are distinguished by the nature of the substrate on the one hand, and by the nature of the pollutants present on the other hand.

The substrates are characterized by meta-descriptors based upon variables such as:
the chemical nature of the mineral and organic constituents;
the water content;
the oxide content;
the pH;
the particle size;
the belonging to one or more mineral classes according to the Strunz classification; and
the redox potential.

The reference substrate may be characterized by physicochemical analyses. It may also be prepared on the basis of predetermined components in order to prepare substrates by assembly.

The reference pollutants are characterized by their chemical composition.

Then, for each of the reference samples, the spectral signature is recorded by subjecting it to illumination by a light source, for example, a xenon lamp, capturing the reflected light and the light emitted by photoluminescence in a wavelength range from thermal infrared to ultraviolet UVC. The data are recorded for each of the samples with an identifier of the reference sample and the physicochemical characteristics.

According to a preferred alternative, the spectral acquisition of the sample or the entire core is carried out, and then one (or more) (sub-)sample(s) is extracted for the physicochemical analysis.

Spectral Acquisition

FIG. 4 depicts the optical diagram of an alternative embodiment of the optical system. The configuration comprises two separate channels used by a fiber bundle connected to a xenon lamp (10) that irradiates soil samples (60) and collects the reflected light in each channel by means of an optical switch (50). A monochromator (51) placed on the optical path provides a secondary beam for exciting the fluorescence.

The first reflectance channel is intended for collecting photons simultaneously on two separate spectrometers:
one spectrometer (61) in the visible and ultraviolet band and
one spectrometer (62) in the infrared band.

The second channel is intended for collecting fluorescence photons in the UV-Visible-NIR range; the monochromatic incident light is selected by means of a monochromator connected to the xenon lamp and the photons are collected on the UV-Visible-NIR spectrometer (61).

1. A method for analyzing soil contamination by pollutants by way of hyperspectral analysis of reflection and/or photoluminescence, wherein the analysis is carried out using a first item of equipment by illuminating a sample using a light source and by at least one spectral sensor sensitive to a spectrum ranging from thermal infrared to ultraviolet, wherein the method includes:
a learning sequence comprising analyzing a plurality of reference samples, and recording in a learning database:
a) the spectral signature of reflection acquired by spectral analysis;
b) known values of the variables representative of the contaminants present in each of the reference samples; and
c) known values of the variables representative of the substrates of each of the reference samples;
a sequence for calibrating an item of field analysis equipment with respect to the first item of equipment, the item of field analysis equipment including a light source and a spectral sensor,
sequences for analyzing a soil sample of a geological site comprising acquiring the reflection and/or photoluminescence signature of the sample using the item of field equipment thus calibrated; and
estimating the characterization of the pollutants by processing the signature by a learning engine exploiting the data from the database established during the learning sequence.

2. The method of claim 1, wherein, during the analysis of a site, at least one core sampling operation is carried out, and wherein the analysis of a plurality of samples distributed over the height of the core is carried out to characterize contaminants at various depths.

3. The method of claim 1, further comprising physically and/or chemically analyzing at least some of the samples.
